(19) United States
(12) Patent Application Publication
(10) Pub. No.: US 2026/0306850 A1
(43) Pub. Date: Oct. 8, 2026

(54) SPINAL CORD INJURY SYSTEMS

(71) Applicant: GLOBUS MEDICAL, INC., AUDUBON, PA (US)

(72) Inventors: Jeshurun S. Paul, Phoenixville, PA (US); David C. Paul, Phoenixville, PA (US); Chad Glerum, Pennsburg, PA (US); Neil R. Crawford, Chandler, AZ (US)

(21) Appl. No.: 19/570,634

(22) Filed: Mar. 18, 2026

Related U.S. Application Data
(63) Continuation of application No. 19/570,409, filed on Mar. 18, 2026.
(60) Provisional application No. 63/782,630, filed on Apr. 3, 2025.

(51) Int. Cl. A61N 1/36; A61N 1/05

(52) U.S. Cl. CPC A61N 1/36139; A61N 1/0553; A61N 1/0556; A61N 1/36062

(57) ABSTRACT

Devices, systems, and methods for treating a spinal cord injury. A spinal cord injury system may include a regenerative device, such as a sleeve, collar, or cuff that fully or partially encircles an injured segment of a spinal cord of a patient. The regenerative device may include a porous or mesh scaffold structure configured to stabilize the local microenvironment, reduce mechanical irritation, and promote axonal regrowth across an injury site. The system may optionally include smart components, such as sensors or electrodes, for diagnostic and/or therapeutic functionality. Alternatively, or in addition, the spinal cord injury system may include a pair of signal transfer devices for communicating signals across the spinal cord injury.

### CROSS REFERENCE TO RELATED APPLICATIONS

This application is a continuation of U.S. patent application Ser. No. 19/570,409 filed on Mar. 18, 2026 which claims priority to Provisional Patent Application No. 63/782,630, filed Apr. 3, 2025, which is incorporated by reference herein in its entirety for all purposes.

### FIELD OF THE INVENTION

The present disclosure relates to medical devices, and more particularly, devices, systems, and methods related to spinal cord injuries and/or restoring function to a patient having a spinal cord injury.

### BACKGROUND OF THE INVENTION

Spinal cord injuries (SCI) are severe, life-altering events that occur when there is damage to the spinal cord, the bundle of nerves that runs down the middle of the spine and carries signals between the brain and the rest of the body. These injuries can result in a partial or complete loss of motor control and sensation below the site of injury, profoundly affecting a person's mobility and sensory functions. Spinal cord injuries can be caused by trauma (e.g., car accidents, falls, sports injuries, and violent incidents), disease (e.g., cancer, arthritis, infections, spinal disc degeneration), or other causes (e.g., ischemia). The symptoms of a spinal cord injury can vary depending on the severity and location of the injury, but can include, for example, loss of movement, loss of sensation, loss of bowel or bladder control, intense pain or stinging, etc. Spinal cord injuries remain a significant medical challenge due to their complex nature and the profound impact they have on a person's life. Current treatment of spinal cord injuries focuses on reducing inflammation, preventing further injury, and managing life-threatening complications.

One present approach for treating chronic back pain and neuropathic pain is spinal cord stimulation. Spinal cord stimulators may include a device that sends electrical pulses directly to the spinal cord to manage pain or block pain signals. The device may emit low-level electrical impulses through leads to the nerve fibers of the spinal cord. These impulses interfere with the transmission of pain signals to the brain, potentially altering the perception of pain and may result in a tingling sensation in the area of pain instead of discomfort.

Spinal cord injury remains a devastating condition associated with permanent neurologic deficits, loss of motor and sensory function, and significant long term morbidity. Treatment strategies tend to focus on decompression, stabilization, and rehabilitation, with limited ability to directly promote axonal regeneration or functional neural recovery. The hostile microenvironment following injury, such as inflammation, scar formation, and inhibitory signaling, can impede meaningful regeneration of neural pathways. There have been some developments in bioengineered biological bridges, for example, made from the patient's own nasal cells or 3D printed scaffolds to bridge severed spinal cords in rats. In addition, there have been developments for peripheral nerves, for example, in the arms and legs to protect repaired nerves from scar tissue and provide a healing environment. Commercially available products such as AxoGuard® and Remplir® effectively function as protective barriers to protect repaired nerves from scar tissue and provide a healing environment. Despite these advances, such products may not be directly applicable to the anatomical, biomechanical, and regenerative challenges associated with spinal cord injuries within the central nervous system.

As such, there remains a significant unmet need for improved systems to protect an injured spinal cord, facilitate nerve regrowth or repair within the spinal canal, and/or restore function so the patient can recover certain bodily functions and/or movements, such as walking, bathroom function, or the like.

### SUMMARY OF THE INVENTION

To meet this and other needs, spinal cord injury systems, devices, and related methods are provided. A spinal cord injury system may include one or more implantable devices configured to facilitate restoration of neural communication and/or support repair of an injured segment of the spinal cord. In some embodiments, the system may include signal transfer components configured to relay neural signals across the injury site. In other embodiments, the system may include a regenerative structure, such as a sleeve or collar, configured to stabilize the injury, promote neural regeneration, and support functional recovery. In yet other embodiments, the system may include a smart implant configured to provide diagnostic monitoring and/or therapeutic functionality to the injured spinal cord. Such implants may include sensors configured to monitor physiological conditions such as motion, strain, temperature, electrical activity, or other factors associated with the spinal cord or surrounding tissues. In some embodiments, the implants may also include stimulation elements, such as electrodes, to provide therapeutic stimulation, and may communicate to external systems for monitoring, analysis, or adjustment of treatment strategies.

In some embodiments, a spinal cord injury system may include a pair of signal transfer devices for communicating signals across the spinal cord injury. In other words, a pair of devices can be utilized to bridge the gap across a spinal cord injury. For example, a first device may be located above a spinal cord injury that sends one or more signals from the patient's brain to a second device located below the spinal cord injury. The signal(s) may communicate certain bodily functions or movements for the person to complete, such as leg movement or bathroom function. The system may include a closed feedback loop, which relays whether the movement or function actually occurred. For example, in the closed feedback loop, the brain of the person communicates with the first device above the spinal cord injury, the first device communicates to the second device below the spinal cord injury, the action or function occurs or not, the second device communicates back to the first device regarding successful or unsuccessful action or function, the first device communicates back to the brain, and the brain takes further action (or not) depending on the outcome. Alternatively, or additionally, an outside system or device, such as a computer, can control transmissions to the first and/or second device with the same type of feedback control. Systems for communicating across spinal cord injuries may help to restore basic bodily functions and enhance the quality of life for patients who have suffered from these severe injuries.

In some embodiments, the spinal cord repair systems may include a regenerative device, such as a sleeve, collar, or cuff configured to fully or partially encircle an injured segment of the spinal cord of a patient. The regenerative device may be positioned external to the dura mater, intradurally in proximity to the spinal cord, in a gap in tissue, or other suitable location, depending on the clinical application and surgical approach. The regenerative device may be formed from a porous mesh, scaffold, or bioengineered material designed to stabilize the local microenvironment and/or guide axonal growth. The structure may provide aligned pathways or microchannels, for example, to promote directional nerve regeneration. In addition, the regenerative device may support vascularization and nutrient diffusion, and may optionally incorporate biologic agents, stem cells, or growth factors to further enhance neural repair. By covering the injured region, the sleeve, collar, or cuff may create a controlled regenerative environment that supports structural protection while facilitating axonal growth and functional recovery.

According to one embodiment, a system for treating a spinal cord injury of a patient includes a first device located above the spinal cord injury and a second device located below the spinal cord injury. The first device transmits neural signals received from a brain of the patient. The second device receives the neural signals transmitted from the first device. The second device sends the neural signals below the spinal cord injury to perform a movement or bodily function as instructed by the brain of the patient. The second device detects whether the movement or bodily function occurs and sends response signals back to the first device. The first device sends the response signals back to the brain, thereby providing closed-loop feedback control across the spinal cord injury.

The spinal cord system may include one or more of the following features. The first and second devices may be implanted into the patient to bridge the spinal cord injury. The first and second devices may include electrodes implanted within a spinal canal or along nerve roots of the patient. The neural signals may be converted to digital data between the first and second devices. The first and/or second device may decode the neural signals from a database compiled for previously mapped brain signals. The first and/or second device may convert the neural signals into electromagnetic waves that can be transmitted through space. The closed-loop feedback control may include artificial intelligence and/or machine learning algorithms.

According to one embodiment, a system for treating a spinal cord injury of a patient includes a first device comprising a first transceiver and first electrode implanted into the patient above the spinal cord injury configured to receive and send neural signals from a brain of the patient, and a second device comprising a second transceiver and a second electrode implanted into the patient below the spinal cord injury configured to receive and send neural signals transmitted from the first device. The second device sends the neural signals below the spinal cord injury to perform a movement or bodily function as instructed by the brain of the patient.

The spinal cord system may include one or more of the following features. The second device may detect whether the movement or bodily function occurs and sends response signals back to the first device. The neural signals from the brain may include motor commands and the response signals may include sensory information. The first device may send the response signals back to the brain to provide closed-loop feedback control across the spinal cord injury. The first and/or second electrode may include a paddle electrode having a paddle with contacts arranged thereon and a lead extending from the paddle. The neural signals may be converted to digital data between the first and second transceivers. The first transceiver may decode the neural signals from a database compiled for previously mapped brain signals. The database may be updated in real-time using artificial intelligence and/or machine learning algorithms. The first and/or second transceiver may convert the neural signals into electromagnetic waves that can be transmitted through space.

According to another embodiment, a method to bypass a spinal cord injury may include one or more of the following in any suitable order: (a) receiving neural signals from a brain of a patient by a first device located above the spinal cord injury and transmitting the neural signals from the first device; (b) receiving the neural signals from the first device into a second device located below the spinal cord injury and transmitting the neural signals from the second device below the spinal cord injury; (c) an action or function occurs or not below the spinal cord injury due to the transmitted neural signals; (d) receiving response signals into the second device after successful or unsuccessful action or function and transmitting response signals back to the first device; and (e) receiving response signals into the first device from the second device and transmitting response signals back to the brain, thereby providing closed-loop feedback to the brain across the spinal cord injury. Prior to receiving signals, the method may include implanting electrodes of the first and second devices, respectively, within a spinal canal or along nerve roots on opposite sides of the spinal cord injury. The method may also include (f) mapping the neural signals to the first and/or second device to recognize the neural signals from the brain to perform the specific action or function; and/or (g) revising the mapped neural signals in real-time based on the successful or unsuccessful action or function.

According to one embodiment, a system for treating a spinal cord injury of a patient includes a regenerative device configured to be implanted adjacent to an injured segment of a spinal cord of a patient. The regenerative device includes a body configured to at least partially encircle the injured segment of the spinal cord, and the regenerative device comprises a porous structure configured to stabilize the local microenvironment surrounding the injured segment of the spinal cord. The regenerative device may be a sleeve, collar, cuff, wrap, or scaffold configured to circumferentially surround the spinal cord. The porous structure may comprise a mesh, lattice, scaffold, fabric, or bioengineered material, for example. The regenerative device may include longitudinal microchannels oriented along a longitudinal axis of the spinal cord. The regenerative device may be configured to be positioned external to a dura mater surrounding the spinal cord. Alternatively, the regenerative device may be configured to be positioned intradurally adjacent to the spinal cord. The regenerative device may include one or more biologic agents, such as growth factors, stem cells, blood, or therapeutic drugs, configured to promote neural regeneration. In some embodiments, the regenerative device may include one or more suture tabs having suture holes configured to receive sutures or other fixation elements to secure the device to local anatomy.

According to one embodiment, a system for treating a spinal cord injury of a patient includes a regenerative device configured to be positioned adjacent to an injured segment of a spinal cord of a patient, the regenerative device including one or more segments configured to at least partially encircle the spinal cord, and a securing member configured to connect ends of the one or more segments to maintain the regenerative device around the spinal cord. The regenerative device may include a single segment and the ends of the segment are joined together by the securing member. Alternatively, the regenerative device may include a first segment and a second segment configured to form a circumferential sleeve when connected. The securing member may include a clip configured to couple the ends of the one or more segments. The securing member may be configured to permit adjustment of a diameter of the regenerative device when positioned around the spinal cord. The one or more segments may include reinforced edges to support connection by the securing member.

According to one embodiment, a method of treating a spinal cord injury may include one or more of the following in any suitable order: (a) accessing an area near a spinal cord of a patient; and (b) implanting a regenerative device adjacent to an injured segment of the spinal cord such that the regenerative device at least partially encircles the spinal cord, the regenerative device including a porous structure configured to stabilize the local microenvironment surrounding the injured segment and/or promote axonal regrowth across the injury site. The method may also include: (c) positioning the regenerative device intradurally adjacent to the spinal cord; (d) positioning the regenerative device external to dura mater surrounding the spinal cord; (e) promoting axonal growth along microchannels in the regenerative device oriented along a longitudinal axis of the spinal cord; and/or (f) adding one or more biologic agents to the regenerative device to promote neural regeneration or support neural repair.

According to one embodiment, a spinal cord repair system includes a smart regenerative device and an external computer platform. The smart regenerative device is configured to be implanted adjacent to an injured segment of a spinal cord of a patient, the smart regenerative device including a body configured to at least partially encircle the injured segment of the spinal cord, the smart regenerative device comprises a porous structure configured to stabilize a local microenvironment surrounding the injured segment of the spinal cord, the smart regenerative device includes one or more neural interface components configured to provide diagnostic monitoring and/or therapeutic functionality and a wireless communication module configured to transmit data from the neural interface components. The external computer platform includes a processor and a memory, the external computer platform is operative to receive data transmitted by the smart regenerative device.

The spinal cord repair system may include one or more of the following features. The neural interface components may include one or more sensors configured to provide diagnostic information related to neural activity, tissue healing, or mechanical loading. The one or more sensors may include a temperature sensor configured to detect temperature changes associated with infection or inflammation, a strain sensor configured to detect deformation or motion near the spinal cord or surrounding tissue, an electrical conductivity sensor configured to detect electrical properties of neural tissue, or an impedance sensor configured to monitor neural tissue properties or signal transmission characteristics. Alternatively, or in addition, the neural interface components may include one or more electrodes configured to deliver electrical stimulation, such as electrical pulses to neural tissue. The neural interface components may include one or more electrodes configured to detect neural activity and/or read nerve signals from neural tissue of the spinal cord.

According to one embodiment, a spinal cord repair system includes a regenerative device and a spinal cord stimulator. The regenerative device is configured to be implanted adjacent to an injured segment of a spinal cord of a patient, the regenerative device including a body configured to at least partially encircle the injured segment of the spinal cord, the regenerative device comprises a porous structure configured to stabilize a local microenvironment surrounding the injured segment of the spinal cord, the regenerative device includes one or more neural interface components configured to provide diagnostic monitoring and/or therapeutic functionality. The spinal cord stimulator is configured to deliver electrical stimulation through the one or more neural interface components. The neural interface components may include one or more electrodes configured to deliver electrical pulses to neural tissue. One or more wires may connect the electrodes to the spinal cord stimulator. The spinal cord stimulator may include a signal generator configured to be implanted subcutaneously within the patient. The signal generator may be configured to transmit electrical therapy signals to the regenerative device to up-regulate and/or down-regulate target nerves. The spinal cord stimulator may include a wireless remote configured to program the signal generator.

According to one embodiment, a method of treating a spinal cord injury may include one or more of the following in any suitable order: (a) accessing an area near a spinal cord of a patient; and (b) implanting a regenerative device adjacent to an injured segment of the spinal cord such that the regenerative device at least partially encircles the spinal cord, the regenerative device includes a porous structure configured to stabilize a local microenvironment surrounding the injured segment and/or promote axonal regrowth across the injured segment, the regenerative device includes one or more sensors and/or electrodes and a wireless communication module configured to transmit and/or receive data. The method may also include one or more of the following: (c) monitoring local conditions via the one or more sensors; (d) applying electrical stimulation through the one or more electrodes; (e) positioning the regenerative device intradurally adjacent to the spinal cord or external to dura mater surrounding the spinal cord; and/or (f) adding one or more biologic agents to the regenerative device to promote neural regeneration or support neural repair.

According to one embodiment, a spinal cord repair system includes a regenerative sleeve and a surgical robotic system. The regenerative sleeve has an inner surface configured to contact or face neural tissue and an opposite outer surface facing away from the neural tissue. The regenerative sleeve includes a flexible material having a porous structure with a network of pores or channels sized and dimensioned to accommodate regenerating axon bundles or neural fibers and provide directional guidance for regenerating axons. The surgical robotic system has a mobile robotic body, including an on-board computer having a processor configured to execute instructions and a memory, a camera electronically coupled to the computer and configured to detect one or more tracking markers and/or anatomical structures, a surgical arm having multiple joints mounted to the robotic body and being moveable based on commands generated by the computer, and an interchangeable end effector removably coupled to a distal end of the surgical arm configured to perform a surgical task. The regenerative sleeve is configured to be implanted adjacent to an injured segment of a spinal cord of a patient via the end effector of the robotic system such that the regenerative sleeve at least partially encircles or covers the injured segment of the spinal cord, and the porous structure is configured to promote axonal growth across the injured segment of the spinal cord.

The spinal cord repair system may include one or more of the following features. The regenerative sleeve may define a generally planar or curved geometry having a first end, a second end opposite the first end, and a pair of lateral side edges extending between the first and second ends. When implanted in the patient, the pores or channels may be oriented parallel to a longitudinal axis of the spinal cord. The regenerative sleeve may be formed of electrospun nanofibers. The regenerative sleeve may be formed of one or more layers to provide different functional properties across a wall thickness of the sleeve. The regenerative sleeve may be formed via additive manufacturing to create a lattice structure configured to promote neural regeneration. The interchangeable end effector may include a robotic hand, guide tube, or other suitable end effector. The robotic system may have a humanoid body with a torso and a pair of articulated legs configured to support and reposition the robotic system, a head coupled to the torso, the head having a pair of cameras electronically coupled to the computer and configured to detect one or more tracking markers and/or anatomical structures, a pair of articulated surgical arms mounted to the torso and electronically coupled to the computer, each surgical arm having multiple joints and being movable based on commands generated by the computer, and the interchangeable end effector removably coupled to a distal end of each surgical arm, each end effector being configured to perform one or more surgical tasks. Alternatively, the robotic system may have a moveable base station housing the on-board computer, a display electronically coupled to the computer, a pair of surgical arms electronically coupled to the computer and movable based on commands processed by the computer, and the interchangeable end effector removably coupled to a distal end of each surgical arm, each end effector being configured to perform one or more surgical tasks.

According to one embodiment, a spinal cord repair system may include a regenerative sleeve and a surgical robotic system. The regenerative sleeve includes a body having a porous structure configured to stabilize a local microenvironment surrounding the injured segment of the spinal cord. The porous structure includes a plurality of pores or channels sized and dimensioned to accommodate regenerating axon bundles or neural fibers and provide directional guidance for regenerating axons. The surgical robotic system has a mobile robotic body, including an on-board computer having a processor configured to execute instructions and a memory, a camera configured to detect one or more tracking markers and/or anatomical structures, first and second surgical arms each having multiple joints mounted to the robotic body and being moveable based on commands generated by the computer, and an interchangeable end effector removably coupled to a distal end of each surgical arm and configured to perform a surgical task. The computer includes a non-transitory computer-readable medium storing instructions that, when executed by the processor, cause the surgical robotic system to: (i) identify a location of an injured segment of a spinal cord of a patient; (ii) generate motion commands to navigate the first surgical arm to align a trajectory to the injured segment of the spinal cord; (iii) control a tool with the second surgical arm to access an area at or near the injured segment of the spinal cord along the trajectory; and (iv) implant the regenerative sleeve with the first or second surgical arm such that the regenerative sleeve at least partially encircles or covers the injured segment of the spinal cord. The regenerative sleeve may be configured to transition from a substantially flat configuration to a curved or cylindrical configuration to accommodate the spinal cord when controlled by the first or second surgical arm of the robot system. The surgical robotic system may be operative to control the first or second surgical arm to secure the regenerative sleeve to dura mater, lamina or bony structures, surrounding tissues, or implanted devices positioned nearby. The surgical robotic system may be operative to adjust placement and tension of the regenerative sleeve during implantation while conforming fit around the spinal cord. The surgical robotic system operates in semi-autonomous or autonomous mode to position the regenerative sleeve relative to the injured segment of the spinal cord.

According to one embodiment, a method of robotic surgery includes one or more of the following in any suitable order: (a) positioning a surgical robotic system adjacent to a patient, the robotic system including an on-board computer having a processor configured to execute instructions and a memory, and configured to execute control, navigation, and task-planning instructions, a camera configured to detect one or more tracking markers and/or anatomical structures, first and second surgical arms electronically coupled to the computer and movable based on commands processed by the computer, and an end effector electronically coupled to each surgical arm; (b) obtaining an imaging dataset of patient anatomy (e.g., acquiring a preoperative or intraoperative imaging dataset of patient anatomy including a computed tomography (CT) scan, magnetic resonance imaging (MRI), and/or fluoroscopic imaging); (c) registering the patient anatomy to the imaging dataset; (d) navigating one end effector of the robotic system to a position and orientation to align a trajectory to an injured segment of a spinal cord of a patient; (e) accessing an area at or near the injured segment of the spinal cord along the trajectory; and (f) implanting a regenerative device adjacent to the injured segment of the spinal cord using one end effector of the robotic system such that the regenerative device at least partially encircles or covers the spinal cord, the regenerative device includes a porous structure configured to stabilize a local microenvironment surrounding the injured segment and/or promote axonal regrowth across the injured segment. The method may further include: (g) selecting the regenerative device based on preoperative imaging data and/or intraoperative sensing; (h) positioning the regenerative device intradurally adjacent to the spinal cord or external to dura mater surrounding the spinal cord; (i) adding one or more biologic agents to the regenerative device to promote neural regeneration or support neural repair; and/or (j) performing a closing procedure using one end effector of the robotic system.

Also provided are kits including implants of varying types and sizes, instruments, and other components for performing the procedures.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 illustrates a patient with a spinal cord injury system including a pair of signal transfer devices for communicating signals across a spinal cord injury according to one embodiment;

FIGS. 2A-2B illustrate a segment of the spine including the vertebrae, intervertebral discs, spinal cord, and nerve roots, and a partial close-up view of a portion of the spinal cord;

FIG. 3 is a representation of the anatomy of the spine;

FIG. 4 illustrates a portion of the spinal cord having an injury that disrupts the flow of axons, and a pair of signal transfer devices for communicating signals across the spinal cord injury in accordance with some embodiments;

FIGS. 5A-5F depict examples of paddle or laminotomy electrodes;

FIG. 6 illustrates a method of communicating signals across the spinal cord injury including a feedback loop, which relays whether the movement or function actually occurred for the patient in accordance with some embodiments.

FIG. 7 illustrates a spinal cord repair sleeve positioned around the spinal cord of a patient in accordance with some embodiments;

FIG. 8 shows a spinal cord repair sleeve having a porous body with reinforced edges and a securing member to close the sleeve in accordance with some embodiments;

FIG. 9 shows a multi-part spinal cord repair sleeve with securing members to complete the sleeve in accordance with some embodiments;

FIG. 10 shows a partial sleeve that partially encircles the injured spinal cord in accordance with some embodiments;

FIG. 11 shows a partial sleeve with optional suture tabs according to one embodiment;

FIG. 12 shows a spinal cord repair sleeve with longitudinal microchannels aligned with the cord axis according to one embodiment;

FIG. 13 shows a smart spinal cord system with integrated sensors, electrodes, or other electronics for diagnostic and/or therapeutic functionality in accordance with some embodiments;

FIG. 14 shows a hardware overview for a smart spinal cord system including a smart spinal cord repair sleeve in communication with an external computer platform in accordance with some embodiments;

FIG. 15 shows a partial spinal cord repair sleeve with integrated electrodes for compatibility with a spinal cord stimulator in accordance with some embodiments;

FIG. 16 is a partial schematic illustration of an implantable spinal cord stimulation system positioned to deliver therapeutic signals to the spinal cord repair sleeve in accordance with some embodiments; and

FIG. 17 shows an example of a humanoid robot and a non-humanoid robotic system for placement of a spinal cord repair sleeve in accordance with some embodiments.

### DETAILED DESCRIPTION OF THE INVENTION

Embodiments of the disclosure are generally directed to devices, systems, and methods related to spinal cord injuries. The spinal cord injury system may include one or more implantable devices configured to restore neural communication and/or promote repair at an injured segment of the spinal cord. The spinal cord injury system may include signal transfer components to transmit neural signals across the injury, a regenerative structure to stabilize the injured area, enhance neural repair, and/or promote functional recovery, a smart implant to provide diagnostic monitoring and/or therapeutic functionality to the injured area of the spinal cord, and/or a spinal cord stimulation system with electrodes to deliver electrical stimulation.

In some embodiments, a spinal cord injury management system may incorporate a set of signal transfer devices configured to bridge the gap caused by a spinal cord injury. Specifically, the system may include a first signal receiver positioned above the site of the injury and a second signal transmitter placed below the injury. For instance, the receiver can receive commands for bodily function or movement from the patient's brain, which are then relayed to the transmitter below the injury such that the signals are transmitted and the person can perform the necessary functions or actions. Additionally, the system may include a comprehensive feedback loop to monitor and confirm the execution of these functions. In this feedback mechanism, brain signals may be sent to the first signal receiver above the injury, the signals are then transmitted to the second transmitter below the injury, which transmits the relevant signals to initiate the desired action. The second transmitter then reports back on the action's success or failure to the first receiver, which communicates to the brain, informing subsequent signals from the brain to adjust the commands accordingly. Alternatively, an external device may oversee this communication, ensuring continuous adjustment and control based on real-time feedback. The system aims to restore and manage critical bodily functions effectively, despite the spinal cord injury.

In some embodiments, the spinal cord repair systems may include a regenerative device, such as a sleeve, collar, or cuff configured to fully or partially encircle an injured segment of the spinal cord. The regenerative device may be positioned external to the dura mater, intradural, or at another suitable location, depending on the clinical application and surgical approach. The regenerative device may be formed from a porous mesh, scaffold, or bioengineered material, for example, designed to stabilize the local microenvironment, reduce mechanical irritation, and modulate inflammatory or scar-forming responses. In some embodiments, the structure may provide aligned pathways or microchannels, for example, to promote directional axonal regrowth across the injury site. In addition, the regenerative device may support vascularization and nutrient diffusion, and may optionally incorporate biologic agents, stem cells, or growth factors to further enhance neural repair. By covering and/or encircling the injured region, the sleeve, collar, or cuff may create a controlled regenerative environment that supports structural protection while facilitating axonal growth and functional recovery.

In some embodiments, the regenerative device may include smart functionality through the integration of one or more sensors, electrodes, electronics, and/or communication components. The smart components may enable the device to monitor physiologic conditions at or near the injury site, such as temperature, strain, motion, pressure, electrical activity, biologic signals, or other signals associated with neural tissue or surrounding structures. The smart device may process or transmit this information to an external system to provide diagnostic insight into healing, inflammation, loading, neural activity, etc. In some cases, the regenerative device may also include therapeutic capabilities, such as delivering electrical stimulation, for example, in combination with a spinal cord stimulator, to further support neural repair and/or functional recovery.

Although generally described for use with spinal cord injuries, it will be readily appreciated by those skilled in the art that the systems and methods described herein may be employed in other suitable applications or other surgical procedures. Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments or modifications thereof are contemplated as falling within the scope of this disclosure and equivalents thereto.

### Spinal Cord Signal Transfer Systems

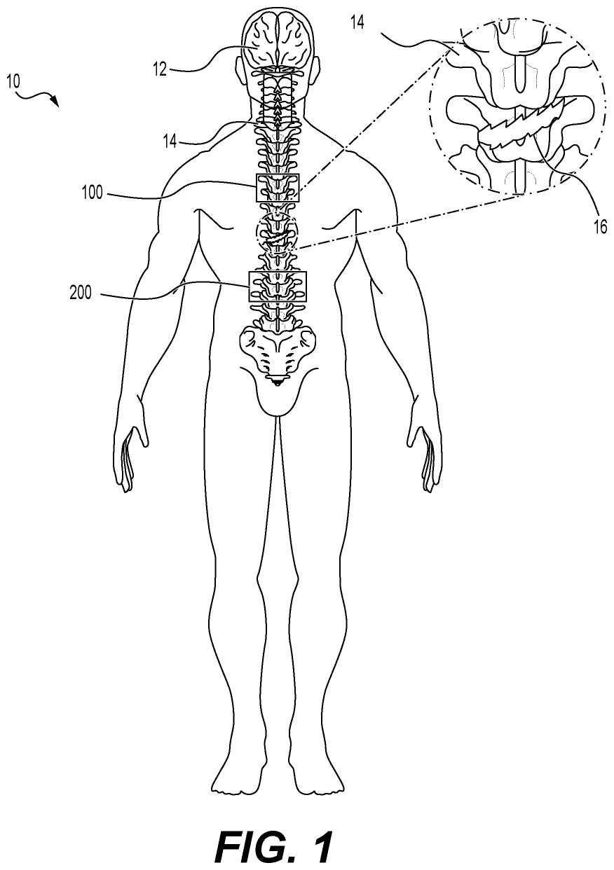

Turning now to the drawing, where like numerals may indicate like elements throughout, FIG. 1 illustrates a person or patient 10 having a spinal cord injury management system according to one embodiment. The patient 10 has a brain 12 and a spinal cord 14 with some type of injury 16. The brain 12 and spinal cord 14 are the two primary components of the central nervous system (CNS) in humans and other vertebrates. They are crucial in processing and transmitting information throughout the body and are responsible for everything from reflexive actions to complex thought processes.

The brain 12 is located in the skull and serves as the control center of the body. The brain 12 is protected by the skull and three layers of membranes called meninges, along with cerebrospinal fluid, which cushions and provides nutrients and removes waste. The brain 12 is responsible for processing sensory information, regulating bodily functions, and facilitating cognition and emotions. The brain is divided into several parts, each with specific functions. The cerebrum is the largest part of the brain, divided into two hemispheres. It performs higher functions such as interpreting touch, vision, and hearing, as well as speech, reasoning, emotions, learning, and fine control of movement. The cerebellum, located under the cerebrum, coordinates muscle movements, maintains posture, and balances. The brainstem acts as a relay center connecting the cerebrum and cerebellum to the spinal cord 14 and controls essential functions such as breathing, heart rate, blood pressure, and digestion.

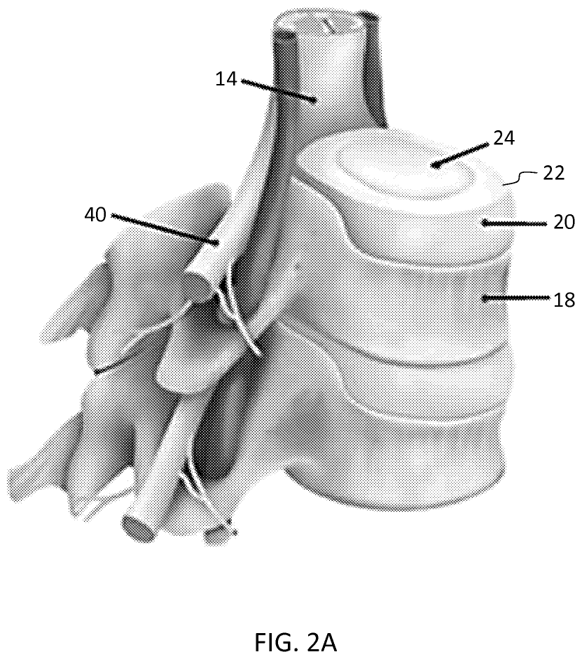

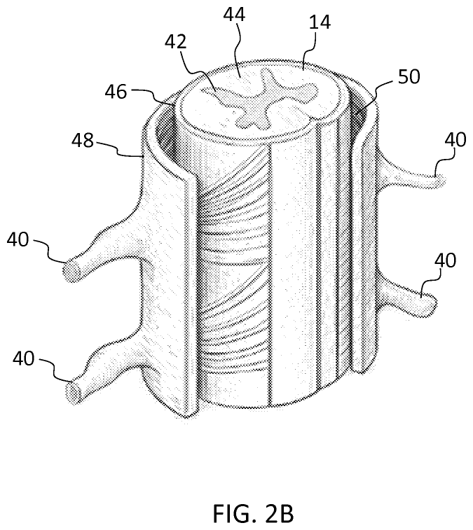

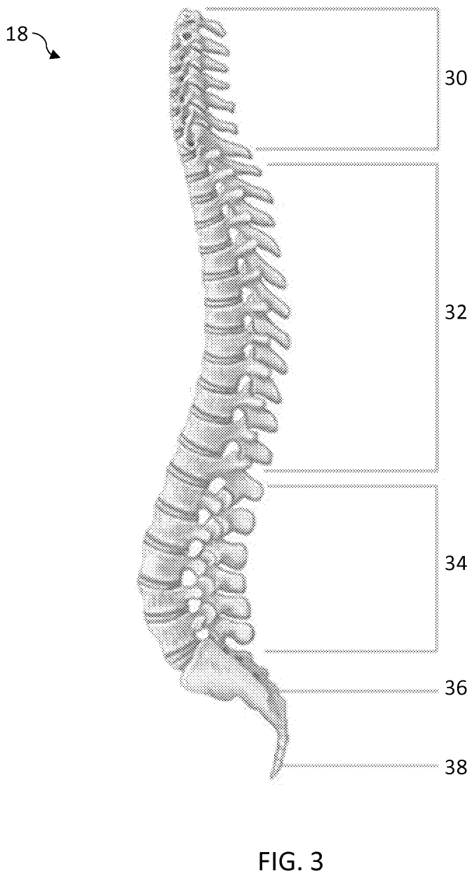

The spinal cord 14 is a long, cylindrical structure of nervous tissue that extends from the brainstem to the lower back, acting as a crucial pathway for transmitting signals between the brain 12 and the rest of the body through the peripheral nervous system. As best seen in FIG. 2A, the spinal cord 14 is located within the vertebral column (backbone) and is protected by the vertebrae 18 of the spine. The vertebrae 18 are separated by intervertebral discs 20, which act as cushions to absorb shock and help maintain flexibility of the spine. The intervertebral discs 20 include an outer annulus fibrosus 22 and an inner nucleus pulposus 24. As best seen in FIG. 3, the human spine is divided into several regions. The cervical region 30, or neck portion, includes seven vertebrae (C1-C7). The thoracic region 32 includes twelve vertebrae (T1-T12). The lumbar region 34 includes five vertebrae (L1-L5). The sacral region 36 includes five fused vertebrae (S1-S5) forming the sacrum. The coccygeal region 38 includes the tailbone. The spine is a central part of the human skeleton, holds up the body and supports all movements, protects the spinal cord 14, and serves as an attachment point for muscles and ligaments.

The spinal cord 14 is a continuation of the brainstem and extends down the back, terminating at the level of the L1-L2 vertebrae 18 in adults. The spinal cord 14 is divided into segments that correspond to the vertebrae 18: cervical 30, thoracic 32, lumbar 34, and sacral 36. Thirty-one pairs of spinal nerves 40 originate from the spinal cord 14, each exiting through an intervertebral foramen (opening between vertebrae 18). The spinal nerves 40 carry sensory and motor information between the central nervous system (brain 12 and spinal cord 14) and the peripheral nervous system (rest of the body). The spinal cord 14 is protected by the vertebral column, the meninges (three layers of protective tissue), and cerebrospinal fluid (CSF). The spinal cord 14 facilitates the transmission of neural signals between the brain 12 and the body. It serves as a major pathway for transmitting signals between the brain 12 and the rest of the body, including sensory information (touch, pain, temperature) and motor commands (movement). The spinal cord 14 also coordinates reflexes independently of the brain 12 through reflex arcs, which allow for rapid responses to stimuli without direct brain involvement.

With further reference to FIG. 2B, a simplified anatomical segment of the spinal cord 14 is shown. The central cylindrical structure represents the spinal cord 14 including gray matter 42 and white matter 44 responsible for transmitting neural signals between the brain 12 and the body. A cross-section at the top of the cord 14 shows the characteristic butterfly-shaped gray matter 42 located centrally, which contains neuronal cell bodies and synaptic connections. White matter 44 surrounds the gray matter 42, which includes ascending and descending nerve tracts that carry sensory and motor signals along the length of the spinal cord 14. Pia mater 46 is the innermost layer of the meninges that envelop the brain 12 and spinal cord 14. Dura mater 48 encases the spinal cord 14 and forms the outermost and toughest protective membrane of the meninges. The dura mater 48 forms a tubular sheath around the spinal cord 14 and helps protect the neural tissue from mechanical injury while maintaining structural containment within the spinal canal. The gap between the dura mater 48 and pia mater 48 contains the arachnoid mater 50, forming the subdural space directly below the dura 48, and the subarachnoid space between the arachnoid and pia 46, which is filled with cerebrospinal fluid (CSF) and blood vessels. The spinal nerve roots 40 extend laterally from the spinal cord 14 and branch outward from the cord 14 to form peripheral nerves that communicate with the rest of the body. These nerve roots 40 carry motor signals from the spinal cord 14 to muscles and sensory signals from the body back to the spinal cord 14 and brain 12. The spinal cord 14, surrounding dura mater 48, and lateral nerve roots 40 form the primary neural pathway responsible for transmitting sensory information and motor commands between the central nervous system and peripheral tissues. The spinal cord 14 ends at the level of the second lumbar vertebra (L2) where it forms a bundle of nerves called the cauda equina that floats in a bag of cerebrospinal fluid.

Depending on the location, type, and severity of the spinal cord injury 16, varying degrees of sensory and motor function loss can occur. To classify and communicate the extent of these injuries, medical professionals use the American Spinal Injury Association (ASIA) Impairment Scale (AIS), which provides a standardized method to assess and describe the injury and its impact on motor and sensory functions. The ASIA Impairment Scale classifies spinal cord injuries into five categories, from A to E, based on the degree of impairment. ASIA A indicates a complete lack of sensory and motor function below the level of injury. No motor or sensory function is preserved in the sacral segments S4-S5. ASIA B indicates some sensory function is retained below the level of injury, but no motor function is preserved more than three levels below the motor level on either side of the body. Sensory but not motor function is preserved below the neurological level and includes the sacral segments S4-S5. ASIA C indicates motor function is preserved below the neurological level, and more than half of key muscle functions below the neurological level of injury have a muscle grade less than 3 (on a scale of 0 to 5). ASIA D indicates motor function is preserved below the neurological level, and at least half of key muscle functions below the neurological level of injury have a muscle grade of 3 or more. ASIA E indicates motor and sensory functions are normal. This classification is used when the patient has recovered completely, but there may have been prior evidence of injury.

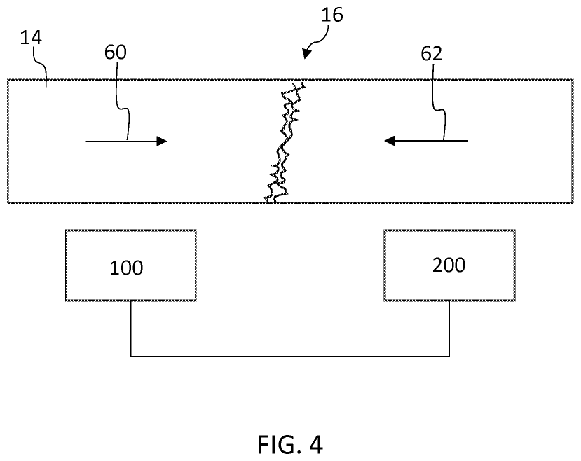

With further emphasis on FIG. 4, in accordance with some embodiments, the spinal cord injury management system may include a pair of signal transfer devices 100, 200 for communicating one or more signals across the spinal cord injury 16. In one embodiment, a first device 100 may be located above the spinal cord injury 16 and a second device 200 may be located below the spinal cord injury 16. The first and/or second devices 100, 200 may include one or more devices configured to transmit, receive, or both transmit and receive signals over a distance.

A signal transmitter may include a device that generates and sends a signal to a receiver. Transmitters convert various forms of information into a signal that can be transmitted through electromagnetic waves, optical, or other transmission media. The transmitter may include an oscillator that generates the carrier wave at a specific frequency, a modulator to modulate the carrier wave to encode the information, an amplifier to boost the strength of the modulated signal, and an antenna that converts the amplified electrical signal into electromagnetic waves that can be transmitted through air or space.

A signal receiver may be configured to detect and process signals transmitted by the transmitter. It captures the incoming wave through an antenna, demodulates the signal to retrieve the encoded information, and then processes it into a form usable for the device. The receiver may include an antenna to capture the incoming electromagnetic waves and converts them back into electrical signals, a tuner that adjusts the receiver to the frequency of the desired signal, filtering out other signals, a demodulator to extract the underlying information from the modulated carrier wave, an amplifier to amplify the weak signals to a level suitable for processing, and a decoder/processor that converts the signal back to its original form.

A signal transceiver combines the functions of both the transmitter and receiver into a single device capable of both sending and receiving signals. The transceivers may include duplex capability, which allows simultaneous or alternating transmission and reception, facilitated by duplexing methods such as time-division duplex (TDD) and frequency-division duplex (FDD). The transceivers may include shared components using common circuit elements for both transmitting and receiving to reduce size, cost, and complexity. The transceivers may include signal processing units with integrated circuits that manage both outgoing and incoming signals efficiently.

In one embodiment, the first device 100 may include a signal receiver that receives one or more signals from the brain and sends those signals to the second device 200. The first device 100 may include a first signal transceiver configured to send and receive signals to/from the second device 200. The second device 200 may include a signal transmitter that receives one or more signals from the first device 100 and transmits those signals past the spinal cord injury 16. The second device 200 may include a second signal transceiver configured to send and receive signals to/from the first device 100.

The signals can correspond to one more signal(s) from the brain 12, which communicate certain bodily functions or movements for the person to complete, such as leg movement or bathroom function. The brain 12 communicates through billions of neurons that transmit signals through axons to different parts of the body. In the spinal cord 14, axons form bundles called spinal tracts within the white matter, facilitating communication between the brain 12 and the body by carrying sensory and motor information. Axons are the primary transmission lines of the nervous system, carrying electrical signals (action potentials) from the neuron's cell body (soma) to other neurons, muscles, or glands. Neural signals are the electrical and chemical communication mechanisms used by neurons (nerve cells) to transmit information throughout the body, enabling the brain to sense, interpret, and act upon the environment. Neurons have a resting membrane potential (a negative charge inside the cell compared to outside). When a neuron is stimulated, this potential changes, leading to an action potential, which is a rapid change in electrical charge that travels down the axon. The brain 12 and spinal cord 14 interact closely as motor and sensory pathways. As shown in FIG. 4, motor commands 60 from the brain 12 can travel down the spinal cord 14 to various body parts, while sensory information 62 from the body is sent back up to the brain 12 through the spinal cord 14. With the presence of spinal cord injury 16, this flow of motor commands 60 and sensory information 62 may be interrupted. By positioning device 100 above the spinal cord injury 16 and device 200 below the spinal cord injury, the flow of commands 60 and sensory information 62 can still be communicated through the system bypassing the injury 16.

The brain signals include electrical impulses traveling through neurons, transmitting information necessary for orchestrating and executing motor functions and other bodily activities. The pyramidal system directly transmits motor impulses from the cerebral cortex to the spinal cord 14. The extrapyramidal system includes a network of neurons that influences and modifies the signals being sent to the muscles. The brain 12 can send signals to the first device 100, for example, related to voluntary movements, reflex actions, and/or autonomic functions. Voluntary movements may include actions like walking, picking up objects, or speaking, which involve precise control from the motor cortex, planning and execution areas in the frontal lobe, and coordination from the cerebellum. Reflex actions are automatic responses to stimuli that can bypass the brain. Reflexes are handled by neural circuits in the spinal cord 14, although the brain 12 can modulate these responses. The brain 12 also sends signals related to the autonomic nervous system, which controls involuntary bodily functions like heart rate, digestion, and respiratory rate. The hypothalamus, along with the brainstem, plays a critical role in regulating these functions.

The first and/or second devices 100, 200 may include one or more electrodes 300 to transmit/receive or otherwise communicate signals with the spinal cord 14, spinal nerves 40, or other biological structures. The electrodes 300 may include percutaneous electrodes that can be inserted into the patient 10 to deliver electrical stimulation or electrical activity, for example, to the spinal cord 14, nerves and/or muscles. In one embodiment, the electrode 300 may be positioned in the spinal canal in the epidural space. Alternatively, a laminotomy may be performed one level below the intended level to allow the electrode to rest at the planned target. The electrodes 300 may be configured to contact or influence myelinated afferent fibers, for example, in the dorsal root, the dorsal root entry zone (DREZ), the dorsal horn, the dorsal columns. The electrodes 300 may also reach the ventral cord or motor neurons, for example, to cause muscular contractions. It will be appreciated that the devices 100, 200 may be configured to communicate with neural and/or nonneural structures in the spinal canal, nerves, or other anatomical structures.

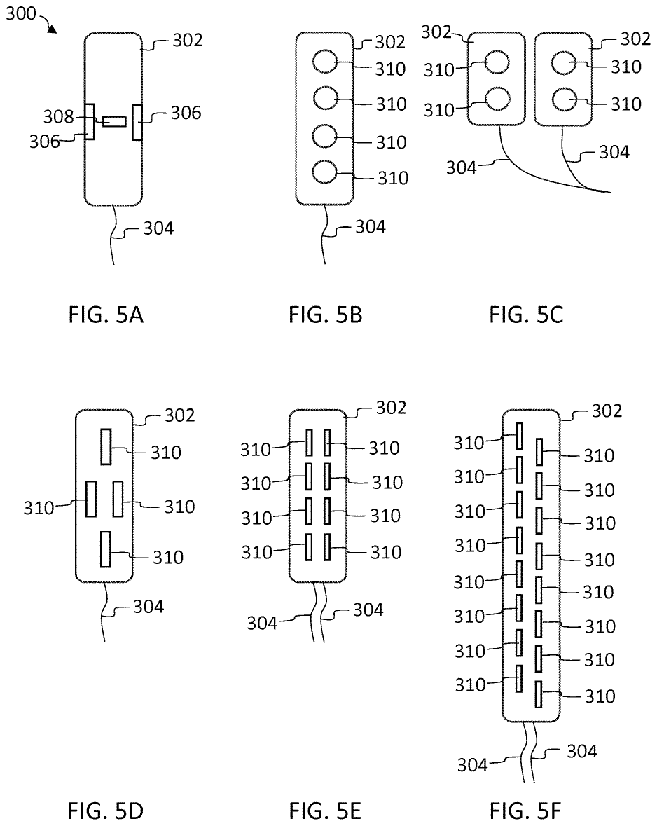

Percutaneous electrodes 300 may include needle electrodes, wire electrodes, epidural electrodes, paddle electrodes, or other suitable electrode types. With further reference to FIGS. 5A-5F, the electrodes 300 may include paddle or laminotomy electrodes, each with one or more paddle(s) 302 and lead(s) 304. The electrode paddles may be characterized by the number and arrangement of the electrodes. For example, electrode paddles may include tripolar, quadripolar, octipolar, dual octipolar, or other suitable configurations. FIG. 5A depicts one example of a tripolar electrode paddle with one paddle 302 having two anodes 306 at the lateral edges with a cathode 308 centrally and a single lead 304. Alternatively, tripolar electrodes may include three electrodes aligned along the paddle for targeted stimulation or electrical activity. FIG. 5B depicts an example of a quadripolar electrode paddle with one paddle 302 having four contacts 310 arranged vertically and linearly with a single lead 304. The electrodes may be in a linear or staggered arrangement to offer greater flexibility in the stimulation pattern or electrical activity. FIG. 5C depicts an example of a quadripolar electrode paddle with two paddles 302 each having two contacts 310 arranged vertically and linearly with two leads 304. FIG. 5D depicts an example of a quadripolar electrode with a single paddle 302 having contacts 310 arranged in a diamond pattern with a single lead 304. FIG. 5E depicts an example of an octipolar electrode paddle with a single paddle 302 having eight contacts 310 arranged in two parallel columns with two leads 304. Alternatively, the contacts 310 may be arranged in two staggered and parallel columns or another suitable arrangement. FIG. 5F depicts an example of a dual octipolar electrode paddle with a single paddle 302 having contacts 310 arranged in two staggered and parallel columns. It will be appreciated that the electrodes may include any suitable number of paddles, contacts, leads, etc. and may be arranged in any suitable manner to convey/receive the signals, stimulation, or other electrical activity.

Different leads may be used to target specific nerves or different nerves depending on the type of spinal cord injury and/or the desired response, such as movement/bodily function in a specific area. In particular, electrodes 300 may be placed to influence certain regions of the body, for example, based on the spinal nerve root 40. Spinal nerves in the cervical region extend to the upper chest and arms, the thoracic spinal nerves innervate the chest and upper abdomen, nerves in the lumbar region travel to the hips, buttocks, and legs, and the sacral nerves provide sensory and motor innervation to the lower limbs, buttocks, and pelvic organs. The spinal nerves carry electrical signals to and from the brain and may cause pain, numbness, tingling or weakness with injury or disease. The table below identifies spinal cord sections and nerve groups:

| Region | Vertebrae | Area of Influence |
|---|---|---|
| Cervical | C1-C3 | Neck muscles |
| | C4 | Diaphragm |
| | C5 | Deltoid (shoulder) |
| | C6 | Wrist |
| | C7 | Triceps |
| | C7-C8 | Fingers |
| Thoracic | T1 | Hand |
| | T2-T12 | Intercostals (trunk) |
| | T7-L1 | Abdominals |
| | T11-L2 | Ejaculation |
| Lumbar | L2 | Hips |
| | L3 | Quadriceps |
| | L4-L5 | Hamstrings - Knee |
| | L4-S1 | Foot |
| Sacral | S2 | Penile erection |
| | S2-S3 | Bowel and bladder |

In addition to the transmitter/receiver and electrode functionality, the devices 100, 200 may optionally include an onboard power source, such as a battery for powering the unit. For example, the battery may include a lithium, nickel-metal hydride, nickel-cadmium, or other suitable battery type. The battery may be rechargeable with an external charging device, for example, with wireless charging capabilities. The devices 100, 200 may be equipped with protection circuits to prevent issues such as overcharging and short-circuiting, which can be hazardous.

The device(s) 100, 200 may optionally include a microcontroller, software and firmware, data storage, sensors, a user interface, communication or telemetry modules, etc. The microcontroller acts as the central processing unit of the device, managing tasks such as signal processing, device control, and communication between the components. The software and firmware may include the basic operating firmware as well as software or data analysis, machine learning algorithms for adaptive responses, and security features to protect patient data. The data storage can include memory components, both volatile and non-volatile, for storing program code, operational data, and logging information about device usage and patient data. Sensors may include pressure, temperature, or motion sensors to monitor or obtain additional information. The user interface may include LEDs or other displays to provide information, such as battery status, operation modes, error messages, or the like. The communication or telemetry modules may include additional communication for different connectivity options like Bluetooth, Wi-Fi, or cellular connections, for example, to enable the device to communicate with external diagnostic, programming equipment, or systems like mobile phones or hospital networks for data synchronization and remote monitoring.

The device(s) 100, 200 may optionally include a digital signal processing (DSP) system. Digital signal processing may convert signals into digital data. The signals may be processed by a DSP chip or other digital processing unit, which may include the use of algorithms. DSP may be used to refine the signals transmitted between the devices 100, 200, enhancing clarity and effectiveness of the transmissions. This can involve filtering noise from the data, amplifying relevant signals, and converting analog signals from the electrodes into a digital form for more precise analysis. DSP may also be used to compress data without losing critical information.

One or more components may be encapsulated in a durable housing or enclosure that protects the device's internal components from physical damage and shields the patient from electrical exposure. The housing or enclosure may be made of biocompatible materials that protect the device from body fluids and prevent the body's immune response from rejecting or reacting negatively to the device. The device(s) may also include an antimicrobial coating or other protections to reduce the risk of infections or other suitable safety features.

The devices 100, 200 (or components thereof) may be implanted into the patient 10 and/or may be used externally to the patient 10. Internal devices may be implanted into the patient 10 along the spine or at any suitable location(s) to bridge the gap of the spinal cord injury 16. For example, internal devices may be attached to specific vertebrae, within or adjacent to the spinal canal to interface directly with the spinal cord 14, implanted near nerve roots 40 or peripheral nerves, or placed at any other suitable location(s). External devices may be able to interface with implanted components. Alternatively, external devices may be configured to send/receive specific transmission signals, generate controlled electric fields that can penetrate into the body to stimulate nerves and muscles, modulate pain signals, or otherwise convey electrical impulses or signals. In the case of controlled electric fields, the fields may be generated by electrodes placed on the skin's surface, targeting specific nerve pathways. If present, internal components can be configured to synchronize with external components to work cooperatively to enhance treatment options.

The devices 100, 200 (or components thereof) may be implanted into the patient 10 by the surgeon and/or with the assistance of computer-assisted technology platforms, such as robotic and/or navigation systems. Surgical robotic systems with integrated navigation may include one or more surgical arms configured to assist a user with one or more surgical tasks, such as placing electrodes 300. End effectors may be attached to each surgical arm to engage instrumentation and perform aspects of the desired surgery. Robots can achieve highly accurate electrode placement at the targeted site. Once electrodes 300 are placed, robotic systems may also assist in initial testing and calibration protocols, adjusting parameters for optimal therapeutic outcomes, or otherwise enhancing the efficacy and safety of such procedures. Examples of surgical robotic and/or navigation systems can be found, for example, in U.S. Patent Publication No. 2019/0021795 and U.S. Patent Publication No. 2017/0239007, which are incorporated by reference herein in their entireties for all purposes.

The brain signals (e.g., neural signals) can be mapped such that the device(s) 100, 200 recognize these signals to perform specific bodily functions and/or movements for the patient 10 to complete. The brain signals may be mapped for the specific person or may be generally mapped for a population. The brain signals may be compiled, for example, in a database such that the device(s) 100, 200 recognize the mapped brain signals. For example, functional brain imaging may be used to help map specific brain signals. Technologies such as functional magnetic resonance imaging (fMRI) and positron emission tomography (PET) may be used to visualize areas of the brain 12 that are active during certain tasks or responses. These imaging techniques can measure changes in blood flow or metabolic activity that correspond to neuronal activity, providing insights into which parts of the brain are involved in specific functions. Electrophysiological techniques may be used to help map specific brain signals. Electroencephalography (EEG) records electrical activity in the brain 12 through electrodes placed on the scalp. Intracranial EEG (iEEG) or Electrocorticography (ECoG) involve electrodes placed directly on the surface of the brain during surgery, and may map brain activity before surgical treatment (e.g., of epilepsy or tumors). Direct Neural Interface/Brain-Computer Interface (BCI) can decode neural signals directly from the brain to control external devices, such as computers or prosthetic limbs, or to communicate. These systems may use machine learning algorithms to correlate patterns of brain activity with specific commands or actions. The motor cortex can be mapped more specifically using techniques like transcranial magnetic stimulation (TMS). TMS can temporarily disrupt or enhance the activity of neurons in specific parts of the motor cortex, helping to identify which areas control specific body parts. Deep Brain Stimulation (DBS) involves implanting electrodes in specific brain areas and is used to treat neurological conditions like Parkinson's disease. It also provides opportunities to study how stimulation of certain brain regions affects motor control and other functions. Neural decoding aims to translate raw neural data into meaningful patterns or commands. This involves understanding the relationship between the firing rates of neurons or neuronal populations and the movements or sensations they represent. It will be appreciated that any suitable technologies and techniques may be used for brain signal mapping for a general population and/or for the given patient 10.

The system and/or device(s) 100, 200 may also incorporate artificial intelligence (AI) and/or machine learning (ML) models. AI and/or ML can be used to determine specific brain signals for the patient, eliminate unnecessary brain signals, update the database, or provide other efficiencies or enhancements for the system. This may include deciphering complex neural signals, adapting patterns based on the patient's response to previous treatments and/or predicting outcomes based on historical data. The AI and/or ML algorithms may include supervised learning, unsupervised learning, semi-supervised, and reinforcement learning algorithms. The AI system may process and analyze large volumes of data, extract insights, predict trends, and learn from new data inputs over time to optimize outcomes. The AI system may continuously monitor its performance and automatically adjust or reorganize data to optimize performance. The AI system may identify patterns, anomalies, and correlations within the data that may not be otherwise apparent. It will be appreciated that any suitable AI algorithms or machine learning models may be used based on the most appropriate methodologies.

In one embodiment, the brain 12 communicates one or more brain signals (e.g., neural signals), for example, including motor command(s) 60. The first device 100 receives, reads, and/or deciphers the brain signals. The first device 100 then transmits one or more of these brain signals, decoded signals, or other suitable signals to the second device 200. The second device 200 receives the transmitted signals from the first device 100 and transmits or otherwise communicates the relevant signals, which continue down the spinal cord 14 past the injury 16. The body can perform the movement or bodily function as intended by the initial brain signals.

Alternatively, an external device can communicate one or more signals directly to the first and/or second device 100, 200. The external device may include a computer or computing unit, for example, including a central processing unit (CPU), memory, and an input/output interface. The central processing unit carries out the instructions of a computer program or software by performing arithmetical, logical, control, and input/output (I/O) operations specified by the instructions. The memory may include volatile and non-volatile memory storage that temporarily or permanently store data and instructions that are currently in use or will be needed by the central processing unit. This may include, for example, random access memory (RAM), read-only memory (ROM), and storage devices like hard drives. It will be appreciated that tangible/non-transitory computer-readable medium comprising software code or storing instructions executable by one or more processors may be adapted, when executed on a data processing apparatus, to perform any computer method set out herein. The input/output interface allows the computer system to interact with the user, take in information, and deliver results, and may include devices such as a monitor, keyboard, mouse, network interface for internet connectivity, and so forth. It will be appreciated that the computer or one or more functions may be replaced or supplemented with other external devices or systems (e.g., cloud computing).

In one embodiment, the external device sends one or more signals to the first device 100 for a desired action or movement. The first device 100 then transmits these signals to the second device 200. The second device 200 receives the transmitted signals from the first device 100 and transmits or otherwise communicates the relevant signals, which continue down the spinal cord 14 past the injury 16. Alternatively, the external device sends one or more signals directly to the second device 200. The body can perform the movement or bodily function as intended by the initial signals.

In an alternative embodiment, the patient 10 may have a prosthetic device, and the second device 200 may communicate signals to control the prosthetic device. The prosthetic device can also communicate signals back to the first and/or second device 100, 200 to determine if the movement or function occurred as intended. AI-enhanced feedback loops may also interpret neural or muscular signals to control prosthetics more accurately, providing a more natural and intuitive use of prosthetic limbs.

The signal system may include a closed feedback loop, which relays whether the intended movement or function actually occurred. After the body receives the signal(s) from the second device 200, the bodily function/movement occurs (or not). The sensory information 62 regarding performance (or lack thereof) may be communicated back to the second device 200. The second device 200 can then transmit the respective signal(s) back to the first device 100 across the injury 16, which can send signals back to the brain 12 (or the external device).

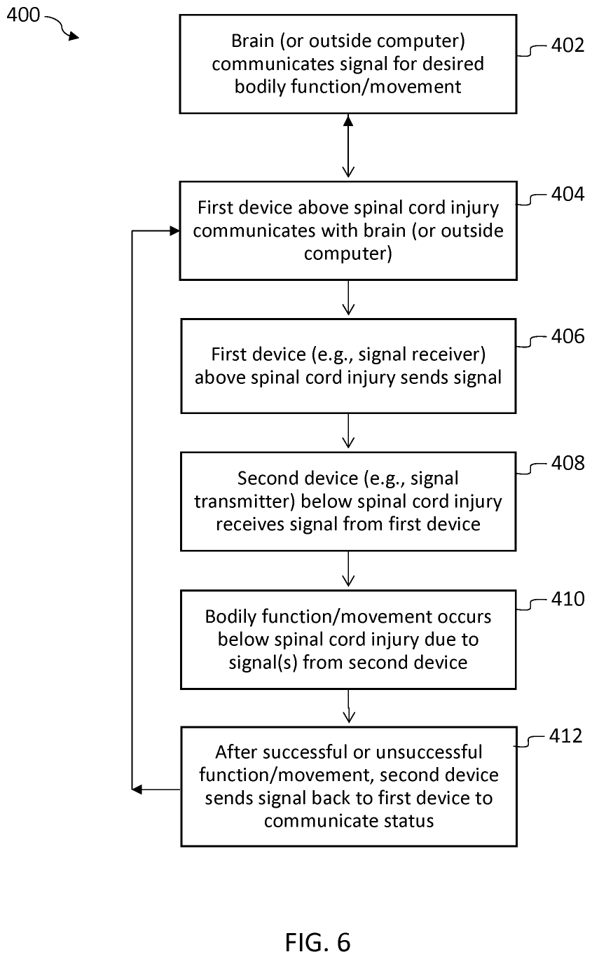

With further emphasis on FIG. 6, in a closed feedback loop 400, the devices 100, 200 bypass the injury 16 and communicate signals above and below the injury 16 back to the brain 12 (or outside computer). In a first step 402, the brain 12 of the person 10 (or outside computer) communicates one or more signals for a desired bodily function/movement. In second step 404, the first device 100 above the spinal cord injury 16 communicates with the brain 12 (or outside computer) including receiving signals for an intended function/action. In third step 406, the first device 100 above the injury 16 communicates to the second device 200 below the spinal cord injury 16, thereby transmitting the received signal(s) to the second device 200. In fourth step 408, the second device 200 below the injury 16 receives the transmitted signals, and further transmits those signals below the spinal cord injury 16. In a fifth step 410, the action or function occurs (or not) below the spinal cord injury 16 due to the signal(s) received from the second device 200. In a sixth step 412, after successful or unsuccessful function/movement, the second device 200 communicates back to the first device 100 regarding successful or unsuccessful action or function. Back at step 404, the first device 100 communicates back to the brain 12 (or outside computer), and the brain 12 takes further action (or not) depending on the outcome.

In this closed feedback loop, the first device 100 located above the injury 16 sends instructions formulated based on the desired bodily functions (like movement or sensory responses) to the second device 200 located below the injury 16. This could involve motor commands for walking or actions like bladder control. Once the signals are executed by the body (or not), the second device 200, below the injury site, captures the outcome of these actions (e.g., whether a movement was successfully performed or not). The second device 200 processes this information and sends it back across the spinal injury to the first device 200. The system can adjust future signals based on this feedback, enhancing accuracy and effectiveness over time. This learning can be facilitated by AI or machine learning that analyzes, modifies and/or optimizes the signal patterns based on real-time results. The system may tailor its functions to the individual needs of the patient 10, adjusting parameters based on the specific responses of the patient 10. AI-enhanced feedback may interpret neural or muscular signals to control movement/function and/or to help in modulating the intensity and pattern of electrical signals based on the feedback received from the patient's motor output and sensory input, effectively creating a personalized response for the patient 10.

### Regenerative Spinal Cord Devices

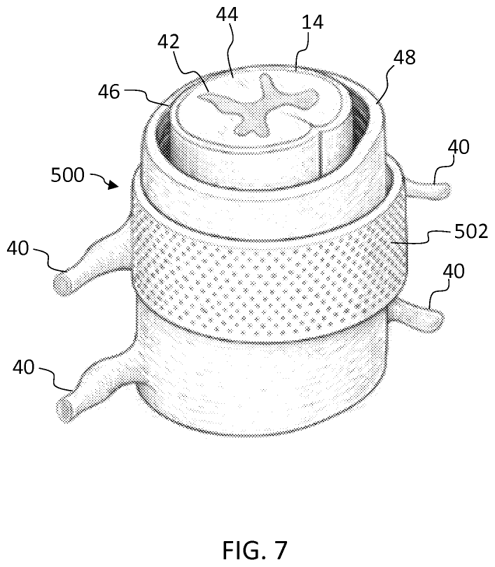

Turning now to FIG. 7, in accordance with some embodiments, the spinal cord repair system may include a regenerative device 500 configured to stabilize the spinal cord injury, promote neural growth or regeneration, and/or support functional recovery. The regenerative device 500 may include a sleeve, collar, or cuff configured to fully or partially encircle an injured segment of the spinal cord 14. The regenerative device 500 may be positioned external to the dura mater 48, intradural, in subdural space 50, adjacent to the pia mater 46, at or near a nerve root 40 or peripheral nerve, in a gap in tissue, or in any suitable location as determined by the surgeon, and depending on the clinical application and surgical approach. By covering and/or encircling the injured region, the sleeve, collar, or cuff may create a controlled regenerative environment that supports structural protection while facilitating axonal growth and functional recovery.

In some embodiments, the regenerative device 500 may have a circumferential or cylindrical body, which may be placed around the dura 48, within the dura 48, or beneath the dura 48, for example. The regenerative device 500 may have a single piece or multi-piece construction. The regenerative device 500 may be formed of a flexible material, which wraps around or drapes over the injured area. The device 500 may wrap back onto itself or may include a clasp, clip, or other securing member 510 to secure the two ends together. Alternatively, the regenerative device 500 may form a partial sleeve to cover all or a region of the injured area. For example, only posterior or lateral portions of the injured spinal cord 14 may be covered by device 500 when using a partial sleeve 500. In some embodiments, the device 500 may be inserted into a resected gap, for example, in the dura 48, to guide axons across the injury.

The regenerative device 500 may be formed from a porous material, mesh, scaffold, or bioengineered material configured to stabilize the local microenvironment surrounding an injured region of the spinal cord and/or guide axonal growth across the injury site. In some embodiments, the material may include a network of pores, channels, fibers, or lattice structures that create an environment for axonal or nerve regrowth. The porous architecture may facilitate cellular infiltration, nutrient diffusion, vascularization, etc. In some cases, the structure may provide directional guidance for regenerating axons via aligned fibers, longitudinal channels, or microstructures. For example, electrospun nanofibers may be oriented to encourage directional regrowth or axonal regeneration. The porous structure may permit fluid exchange, cellular infiltration, and tissue integration while maintaining sufficient structural integrity to provide mechanical support and positional stability around the spinal cord 14.

In some embodiments, the regenerative device 500 may include a single or multi-layer structure to provide different functional properties across the thickness of the device 500. A single layer may include the porous mesh, lattice, scaffold, or structure that provides mechanical support, enhanced tissue interaction, and promoted neural regeneration. Alternatively, multiple layers may be stacked together to perform complimentary functions. For example, the device 500 may include an outer porous layer and an inner nanofiber layer. The inner layer may be positioned adjacent to the neural tissue, for example, to mimic aspects of an extracellular matrix to promote cell attachment, migration, and differentiation. Different or additional layers may be used depending on the desired biologic or mechanical characteristics.

In some embodiments, the regenerative device 500 may be manufactured using additive manufacturing techniques such as three-dimensional (3D) printing to create precise porous or lattice structures tailored to promote neural regeneration. Alternatively, the material may include woven or knitted meshes, braided structures, perforated sheets, or fabric-like materials able to conform to the surface of the spinal cord 14. Suitable materials may include biocompatible polymers, elastomers, hydrogels, or composite materials that provide mechanical compliance with surrounding neural tissues. In certain embodiments, the regenerative device may be formed from bioresorbable materials that gradually degrade as neural regeneration occurs. Alternatively, the device 500 may be formed from durable biostable materials intended for long term implantation. In some embodiments, the device 500 may incorporate conductive materials or conductive pathways configured to support electrical stimulation, neural recording, or signal transmission. In further embodiments, the regenerative structure may be composed of or incorporate biologic materials such as extracellular matrix components, collagen scaffolds, allografts such as decellularized allograft tissues, or other biologically derived materials that further enhance cellular attachment and axonal growth.

The regenerative device 500 may optionally incorporate biologic agents, stem cells, and/or growth factors to further enhance neural repair and improve the healing environment at the site of the injury. Such agents may include stem cells, blood, growth factors, therapeutic drugs, biologics, or other regenerative material delivered locally by the device 500. The agents may permit controlled or sustained release over time. For example, the device 500 may deliver growth factors that promote axonal regeneration, anti-inflammatory agents that reduce local inflammation, or compounds that inhibit glial scar formation. In some embodiments, the device may also support or carry stem cells or other regenerative cells that contribute to neural repair, tissue regeneration, and functional recovery.

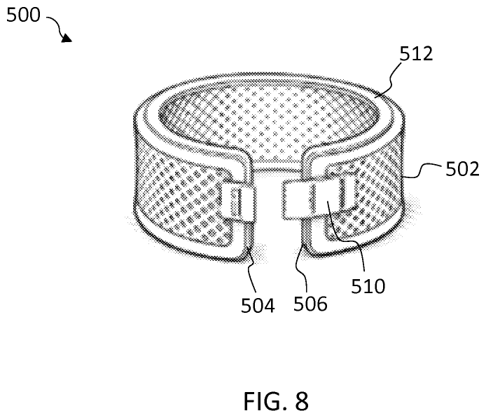

With further reference to FIG. 8, one example of regenerative device 500 is shown in more detail. In this embodiment, the circumferential regenerative sleeve 500 is configured to fully encircle the spinal cord 14 or dura mater 48. The sleeve 500 extends from a first end 504 to a second end 506, which may be secured together via securing member 510. The securing member 510 may include tabs, straps, coupling members, or fastening mechanisms extending from one or both ends 504, 506 of the sleeve 500. The securing member 510 may interlock, buckle, clip, snap, or otherwise engage to hold the sleeve 500 in a circumferential configuration after implantation. The securing member 510 may permit the surgeon to adjust the tightness or diameter of the sleeve 500 and maintain proper alignment around the injured region.

The sleeve 500 may include a reinforced outer edge 512 surrounding the inner porous mesh region 502. The outer edge 512 may form a partial or continuous border, which may be mechanically reinforced relative to the mesh interior 502 to provide additional strength and dimensional stability. In some embodiments, the outer edge may serve as a structural support for optional anchors, for example, to the dura 48, lamina, pedicles, or other fixation implants (e.g., bone screws).

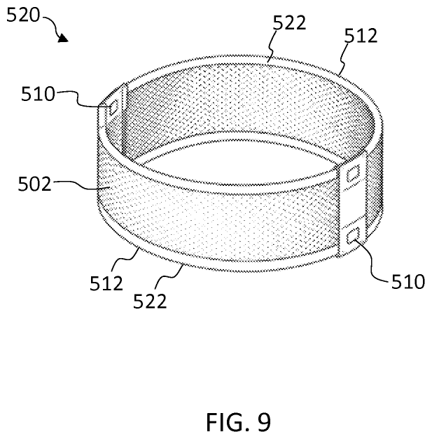

Turning now to FIG. 9, a multi-part regenerative sleeve 520 is shown according to one embodiment. In this embodiment, the multi-part sleeve 520 may be divided into two or more complimentary segments 522 that, when assembled, form the cylindrical collar. Each segment 522 may include a curved mesh body 502 with reinforced perimeter edges 512 similar to sleeve 500. During implantation, the segments 522 may be coupled together to encircle the cord 48. The opposing edges may include securing members 510, such as tabs, slots, clips, buckles, etc. that allow the surgeon to secure the segments 522 together. It will be appreciated that any suitable number of segments 522 may be used to complete the sleeve 520.

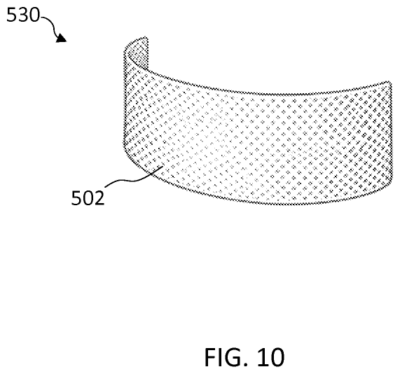

Turning now to FIG. 10, a partial sleeve 530 is shown according to one embodiment. The partial sleeve 530 may be comprised of a flexible mesh body 502, which may be configured to conform to fit the desired area. In some embodiments, the partial sleeve 530 may be curved, semi-cylindrical, or otherwise contoured to fit over or cover a selected portion of the spinal cord 14, surrounding dura mater 48, or other anatomical area, without completely encircling the structure. The flexible nature of the mesh 502 may allow the device to adapt to variations in patient anatomy and accommodate subtle movements while maintaining stable positioning over the treatment region.

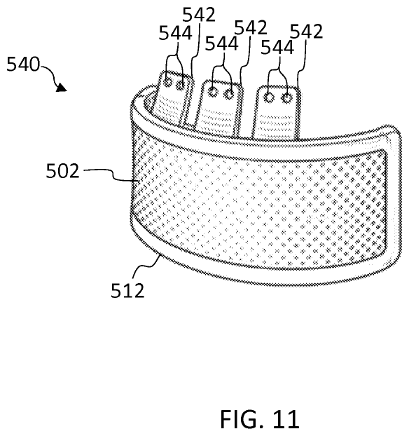

Turning now to FIG. 11, a partial sleeve 540 with suture tabs 542 is shown according to one embodiment. The partial sleeve 540 may include mesh body 502 with reinforced perimeter 512. One or more suture tabs 542 may extend outward from the perimeter edge 512, in any direction, to facilitate fixation of the device to the surgical site. The suture tabs 542 may be integrally formed with the sleeve 540 or may be attached thereto. Each suture tab 542 may define one or more suture holes 544 configured to receive sutures, stables, barbs, micro-hooks, clips, anchors, or other attachments. The partial sleeve 540 may be anchored, for example, to the dura 48, the lamina or other bony structures, surrounding tissues, or implanted devices positioned nearby. The surgeon may adjust the placement and tension of the sleeve 540 during implantation while conforming fit around the patient's anatomy.

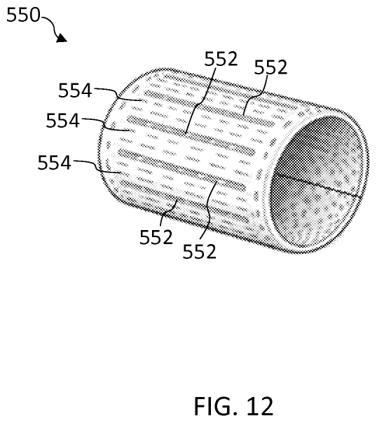

Turning now to FIG. 12, a circumferential regenerative sleeve 550 is shown in accordance with one embodiment. In this embodiment, the mesh is replaced with a series of longitudinal microchannels 552 extending along the length of the device. The longitudinal microchannels 552 may be oriented substantially parallel to the long axis of the spinal cord 14 and may be dimensioned to accommodate regenerating axon bundles or neural fibers. The channels 552 may extend continuously along a length of the sleeve 550 or may include segmented or interconnected channels. In addition, the sleeve 550 may include elongated perforations 554 extending through the wall of the sleeve 550. The perforations 554 may provide pathways for fluid exchange, nutrient diffusion, vascular infiltration, and cellular migration between the surrounding tissues and the interior of the sleeve 550. The structure may provide aligned pathways or microchannels, for example, to promote directional nerve regeneration across an injured region of the spinal cord 14. It will be appreciated that the mesh structure 502, microchannels 552, perforations 554, and/or other configurations may be used with any of the embodiments described herein. The regenerative sleeves may help to stabilize the injury, reduce scar formation, and/or support axonal regrowth that correspond to the natural orientation of the spinal cord nerve fibers.

In another embodiment, the regenerative sleeve 550 may be configured as a 3D-printed scaffold or decellularized tissue matrices. These materials including but not limited to PCL (poly-caprolactone) and processed nerve allografts, provide a physical and trophic bridge that supports neuronal survival, directed axonal growth and provides support structure as a bridge over lesions. The sleeve 550 may be configured to include stem cells, neural progenitor cells, and/or neurotrophic factors (BDNF, NGF) to stimulate nerve growth. The sleeve 550 also provides the a structure support over the lesions sites, to reduce glial scar formation. In another embodiment, as sleeve 550 may be configured with a material to be reabsorbed over a period of time, as the nerve is generated and normal nerve function returns to a patient.

### Smart Spinal Cord Devices

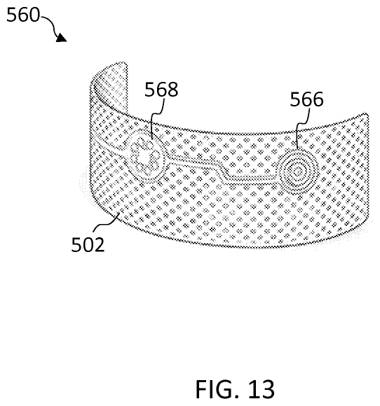

Turning now to FIG. 13, a smart regenerative device 560 is shown according to some embodiments, which incorporates diagnostic and/or therapeutic technologies into the device 560. The regenerative sleeve 560 may be the same or similar to sleeve 530 or any other suitable device, while being equipped with smart capabilities to detect, monitor, and/or actively control the environment around the installed device. For example, the smart device 560 may be configured to detect infection, monitor motion, accelerate axonal regrowth, etc. The smart device 560 may incorporate electronic components, such as sensors, gauges, electrodes, printed circuit boards, batteries, and power/data transmission components to detect, monitor, collect, and transmit/receive relevant data. The smart device 560 may communicate with one or more external platforms, such as a patient wearable device, external computer, cell phone, or other monitoring/communication system to provide data to the surgeon, patient, clinician, or other members of the care team. The smart implant data may be communicated for clinical decision making, to make adjustments in situ, and/or to populate diagnostic or predictive algorithms. The smart device 560 may enhance patient outcomes by minimizing the risk of infection, monitoring local conditions, and/or accelerating axonal regrowth.

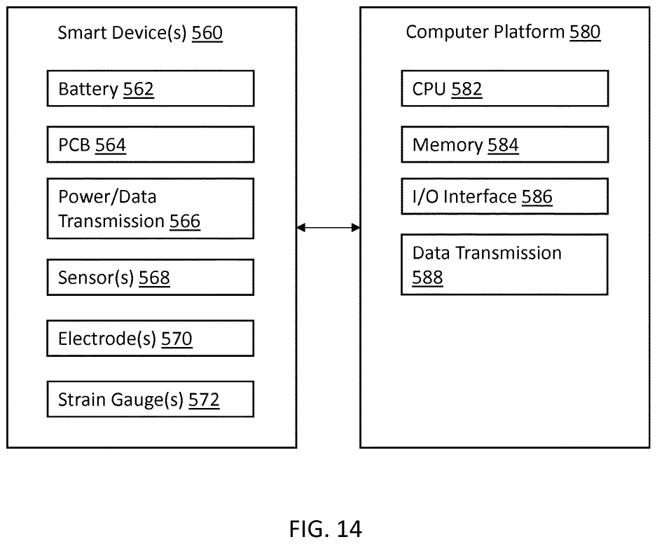

FIG. 14 illustrates a block diagram of the architecture for a smart spinal cord repair system in accordance with some embodiments. The smart spinal cord repair system may include one or more smart devices 560 and an external computer platform 580. The smart sleeve 560 may include, for example, an on-board battery 562, printed circuit board (PCB) 564, power/data transmission component 566, sensor(s) 568, electrode(s) 570, and/or strain gauge(s) 572. The battery 562 may include a rechargeable micro-cell or thin-film pack (e.g., Li-poly cell), for example, which powers the internal electrical components.

The printed circuit board 564 may include a miniature printed circuit board that carries a microcontroller (MCU) and associated circuitry. The printed circuit board 564 may also include volatile and non-volatile memory storage that temporarily or permanently store data and instructions that are currently in use or will be needed by the microcontroller. This may include, for example, random access memory (RAM), read-only memory (ROM), or electrically erasable programmable read-only memory (EEPROM). The printed circuit board 564 may also include an analog-to-digital converter (ADC), power conditioning, wireless transceiver, and/or other relevant components.

The power/data transmission component(s) 566 may include an inductive coil, radiofrequency (RF), optical link, or other suitable components that can recharge battery 562 and/or shuttle telemetry/commands to/from the computer platform 580. The power/data transmission component(s) 566 may include a bidirectional wireless transceiver and inductive power receiver, such as a multi-turn coil configured to receive inductive power from an external transmitter and serve as an antenna for bidirectional data exchange. The sensor(s) 568 may include, for example, temperature probes, thermistors, thermocouples, or digital temperature sensor integrated circuits (IC), or other sensor capable of detecting thermal variations in the local tissue environment. The sensor(s) 568 may include motion and/or vibration sensing technologies, such as motion sensors calibrated relative to Earth's gravity vector, accelerometers, inertial measurement units (IMUs), magnetometers, tilt sensors, strain-based motion sensors, optical motion sensors, displacement sensors, or other sensing elements capable of detecting motion, orientation, vibration, or acceleration. The electrodes 570 may include impedance-spectroscopy electrodes (e.g., with potentiostat subsystem) and/or electrical stimulation electrodes, for example. In one embodiment, the electrodes 570 may be configured to stimulate and/or read nerve signals. The load or strain gauge(s) 572 may include one or more foil strain gauges, semiconductor strain gauges, thin-film strain gauges, microelectromechanical systems (MEMS) strain sensors, piezoresistive strain gauges, or other deformation sensing elements configured to measure load and/or micromotion.

The external computer platform 580 may include a central processing unit (CPU) 582, memory 584, input/output (I/O) interface 586, and/or data transmission 588. The CPU 582 can execute firmware for data logging, analytics, and closed-loop control. The memory 584 may include non-volatile storage, for example, for raw sensor data, calibration tables, and event logs. It will be appreciated that tangible/non-transitory computer-readable medium comprising software code or storing instructions executable by one or more processors may be adapted, when executed on a data processing apparatus, to perform any computer method set out herein. The I/O interface 586 may include ports for surgeon console, hospital network, or cloud gateway. The data transmission 588 may include a wireless module (e.g., transceiver) that can maintain a bidirectional link with the smart device 560. It will be appreciated that the external computer platform 580 may include devices, such as patient wearable devices, external computers, tablets, cell phones, cloud computing, or other monitoring/communication systems to interact with the surgeon, patient, clinician, or other members of the care team.

The bidirectional arrow between the smart devices 560 and computer platform 580 shows that real-time telemetry (e.g., temperature, EIS spectra, load, battery status, etc.) can be streamed to the external platform 580 for processing, display, and/or archival purposes. In return, the computer platform 580 can provide data to the smart device 560, for example, to adjust stimulation waveforms, alter sampling intervals, or trigger high-resolution data bursts. The data transfer can occur at specific times, for a given duration, or continuously, enabling diagnostics and patient therapy over time.

The smart sleeve 560 may provide diagnostic and/or therapeutic functionality. For example, the sleeve 560 may incorporate temperature sensors 568 configured to detect localized temperature changes that may indicate infection or inflammation. The sleeve 560 may include strain sensors 572 capable of monitoring spinal cord motion, deformation, or mechanical forces acting on the tissue. In addition, electrical conductivity sensors 568 or impedance monitoring elements may be incorporated to assess neural tissue properties, signal transmission characteristics, or changes in the electrical environment surrounding the spinal cord 14. These sensing capabilities may provide diagnostic information related to healing progression, neural function, or potential complications following implantation.

### Integrated Spinal Cord Stimulation

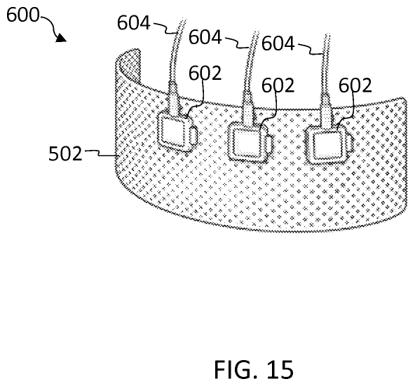

Turning now to FIG. 15, a smart regenerative device 600 is shown, which incorporates electrodes 602 for spinal cord stimulation in accordance with some embodiments. The regenerative sleeve 600 may incorporate one or more electrodes 602 configured to provide electrical stimulation to or near the spinal cord 14 and/or neural tissue. In this embodiment, the regenerative structure may function as a scaffold for neural repair as well as an active neuromodulation device. The electrodes 602 may be integrated into, embedded in, or supported by the body of the sleeve 602. The electrodes 602 may be formed from conductive biocompatible materials such as platinum, platinum-iridium, conductive polymers, etc. The electrodes 602 may be arranged circumferentially, longitudinally, or distributed in arrays along the device 600 to deliver electrical stimulation to selected regions of the spinal cord 14. The electrodes 602 may be arranged to allow stimulation above, below, or across the injured spinal segment. The electrodes 602 may be configured to deliver electrical pulses to neural tissue that stimulate neural pathways, facilitate signal transmission across an injury site, or enhance neural plasticity and regeneration.

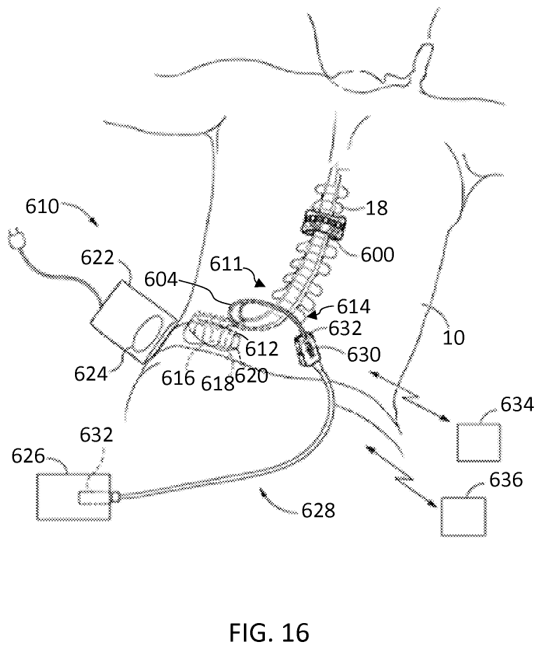

The regenerative sleeve 600 may function in combination with a spinal cord stimulator 610 in accordance with some embodiments. The electrodes 602 may include leads or wires 604 to connect to the sleeve 600 to the spinal cord stimulator or other neuromodulation device. FIG. 16 schematically illustrates a representative spinal cord stimulator or spinal cord treatment system 610 for treating one or more neurologic diseases. The spinal cord treatment system 610 can include a signal delivery system 611 having a signal generator 612 (e.g., a pulse generator) and a signal delivery device comprising one or more signal delivery elements (e.g., electrodes 602 on sleeve 600). The signal generator 612 can be connected directly to the sleeve 600, or it can be coupled via a signal link 614 (e.g., an extension). In some embodiments, the signal generator 612 may be implanted subcutaneously within the patient 10. The signal generator 612 and/or other elements of the treatment system 610 can include one or more processors 616, memories 618, and/or input/output devices 620. The signal generator 612 can transmit signals (e.g., electrical therapy signals) to the sleeve 600 (e.g., electrodes 602) that up-regulate (e.g., stimulate or excite) and/or down-regulate (e.g., block or suppress) target nerves (e.g., local vagal nerves). In some embodiments, the signal generator 612 can obtain power to generate the therapy signals from an external power source 622. The external power source 622 can include an external coil 624 that communicates with a corresponding internal coil (not shown) within the implantable signal generator 612.

During at least some procedures, an external programmer 626 (e.g., a trial modulator) can be coupled to the sleeve 600 with a cable assembly 628, for example, connected to connector 630 to vary the modulation parameters. The signal generator 612, lead extension, external programmer 626 and/or connector 630 can each include a receiving element 632 to facilitate a simple coupling and decoupling procedure between the elements. In some embodiments, the signal delivery parameters provided by the signal generator 612 can be updated after the signal generator 612 is implanted, via a wireless physician's programmer 634 (e.g., a physician's remote) and/or a wireless patient programmer 636 (e.g., a patient remote). Further details regarding spinal cord stimulation are provided in U.S. Pat. Nos. 11,446,504 and 11,801,382, which are incorporated by reference herein in their entireties for all purposes.

The regenerative sleeve with integrated electrodes combines structural neural repair with active neuromodulation in a single implantable device. The integrated electrodes reduce the need for separate stimulation hardware. The sleeve functions as a scaffold that stabilizes the injury and promotes axonal regeneration as well as an electrical stimulation platform to deliver targeted pulses to neural tissue. The device may stimulate selected or localized regions of the spinal cord to enhance neural plasticity, facilitate signal transmission across damaged pathways, and promote functional recovery.

### Robot Placement of Spinal Cord Devices

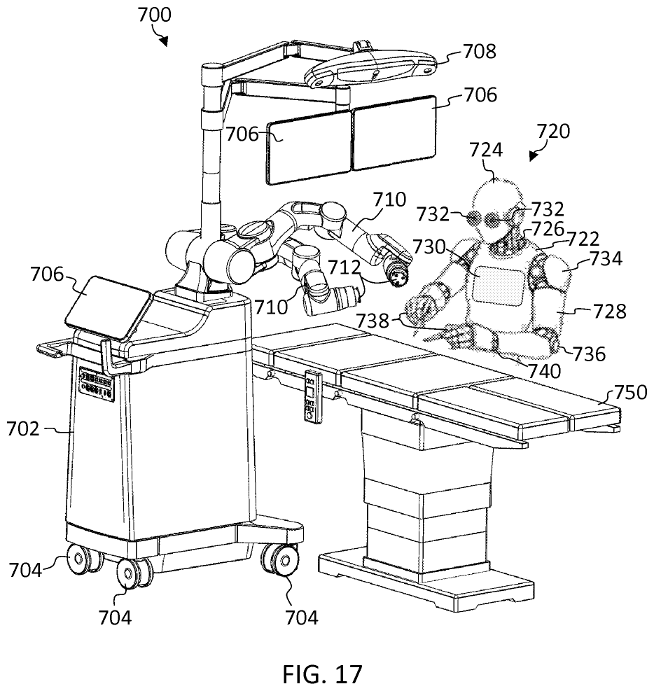

Turning now to FIG. 17, examples of robotic and/or navigation systems are shown in accordance with some embodiments. The robotic systems may include one or more non-humanoid surgical robots, such as mobile robotic platform 700, humanoid surgical robots 720, or any suitable combination of robotic and/or navigation systems. In some embodiments, one or more robotic systems 700, 720 may be used to position, place, and/or install the spinal cord device(s). In some embodiments, the robotic system may operate in a robot-assisted configuration in which the robot provides guidance, positioning, stabilization, or instrument manipulation under the control or supervision of a surgeon. In other embodiments, the robotic system may operate in a semi-autonomous or fully autonomous mode in which the robot performs one or more surgical tasks based on preoperative planning, navigation data, and/or intraoperative sensing.

In some embodiments, the surgical robot includes mobile robotic platform 700 having a robotic base station 702. The robotic base station 702 may have a mobile cabinet or portable frame, for example, on casters or wheels 704, which houses an on-board computer or computing unit for controlling functionality of the robotic system 700. The robotic system 700 may include displays or monitors 706, which may be operated by one or more surgeons, robots, or other users. The robotic system 700 may utilize a navigation or optical camera system 708, such as one or more infrared cameras (e.g., bifocal or stereophotogrammetric cameras) or other suitable cameras or sensing technologies. The robotic system 700 includes one or more surgical robotic arms 710 to perform a wide range of motions and adjustments. The distal end of each surgical arm 710 includes an end effector interface 712 for securing an end effector (e.g., robotic hand end effector 738, guide tube, etc.) to perform a surgical task. Further details on such surgical robotic systems are provided in U.S. Publication No. 2025/0288370, which is incorporated by reference herein in its entirety for all purposes. It will be appreciated that any suitable surgical robot systems and/or navigation systems may be used to complete the desired surgical tasks.

In some embodiments, the surgical robot includes a humanoid or anthropomorphic multi-arm robot 720. Surgical robotic system 720 may include, for example, a body or torso 722 housing one or more primary system components, a head 724 mounted on a neck assembly 726 on the torso 722, one or more articulated upper limbs or arms 728 each supporting an end effector assembly (e.g., end effector robotic hand 738), and a pair of articulated lower limbs or legs supported on feet (not shown). The torso 722 may support and house primary system components, such as one or more processors, power management circuitry, communication modules, and control electronics. The robot 720 may include one or more user interfaces, such as displays or monitors 730 including touchscreen displays, which may be operated by one or more surgeons or other users. The robotic system 720 may include a head assembly 724 configured to support one or more sensing and perception devices, such as a pair of cameras 732, which function as the eyes for the robot. The cameras 732 may include infrared cameras, machine vision cameras, optical cameras, or other imaging or sensing devices. The robotic system 720 may include one or more articulated upper limbs or arms 728 configured to manipulate end effectors or surgical tools. Each surgical arm 728 may include a plurality of arm segments or links interconnected by various types of joints. For example, each arm 728 may include multiple joints, such as shoulder joint 734 and elbow joint 736, providing multiple degrees of freedom to enable precise positioning, orientation, and reach within the surgical workspace. Distal portions of the surgical arms 728 may include a wrist or end effector interface 740 configured to receive interchangeable end effectors, such as robotic hands 738, guide tubes, graspers, imaging probes, retractors, suturing devices, drills, cutting instruments, or other surgical instruments, allowing the robot 720 to perform a variety of surgical tasks.

As shown, system positioning at the operating room table 750 is flexible and may be based on preference for a given procedure. One or more robotic systems 700, 720 may be positioned next to the patient 10 or toward the foot or head of the patient 10. The robotic system 700, 720 may stand next to or across from the surgeon or other clinical staff, allowing the robot 700, 720 to operate cooperatively within the surgical team without obstructing clinician access to the patient 10. In some embodiments, the robot 700, 720 may reposition itself during the procedure to accommodate different phases of surgery, changes in patient positioning, or tool exchanges.

Before or during the medical procedure, two-dimensional (2D) and/or three-dimensional (3D) images, such as computed tomography (CT) scans, magnetic resonance imaging (MRI), and/or fluoroscopic imaging, may be taken of a desired surgical area of a patient and provided to the on-board computer(s) of the robot system(s) 700, 720. The robot and/or surgeon may use the images to program a desired point of insertion and trajectory for one or more surgical instruments to reach a desired anatomical target within or upon the body of the patient. The desired point of insertion and trajectory may be planned on the images, which may be displayed on monitor(s) 706, 730. The systems may include 2D & 3D imaging software that allows for preoperative planning, navigation, and guidance throughout the surgical procedure. Further details of surgical robotic and navigation systems can be found, for example, in U.S. Patent Publication No. 2019/0021795 and U.S. Patent Publication No. 2017/0239007, which are incorporated herein by reference in their entireties for all purposes.

In some embodiments, a method of installing the regenerative sleeve may include utilizing the robotic surgical system 700, 720 to assist with positioning and/or placement of the device at or near the injured spinal cord segment. Regenerative device 500 will be described with reference to the following embodiment, although it will be appreciated that any of the spinal cord device(s) 100, 200, 300, 500, 520, 530, 540, 550, 560, 600 may be used with the robotic systems. During the surgical procedure, one or more surgical robotic arms 710, 728 may be configured to position, place, and/or install the spinal cord device 500. For example, the robotic system 700, 720 may align surgical instruments, position the regenerative sleeve 500 relative to the spinal cord 14, and/or assist in securing the device 500 at the surgical site (e.g., via sutures). In one embodiment, the robotic system 700, 720 may first register the patient anatomy using imaging, navigation markers, and/or intraoperative sensing to identify the location of the spinal cord and/or injury site. Based on the registration and preoperative and/or intraoperative plan, one or more robotic arms 710, 728 may guide surgical instruments (e.g., via robotic hand end effector 738 or guide tube) to expose or access the target area (e.g., posterior and/or lateral portions of the injured spinal cord 14). The robot system 700, 720 may position the regenerative sleeve 500 adjacent to the spinal cord 14 and may manipulate the sleeve 500 using the end effector 738 or surgical instrument to place the sleeve 500 around, against and/or within the dura mater 48 or spinal cord 14. If the sleeve 500 requires assembly or manipulation, the robotic system 700, 720 may assist in wrapping, assembling the sleeve components, or closing the securing member(s) 510, for example. If desired, the system 700, 720 may also assist in securing the device, for example, using sutures, clips, or other fixation mechanisms. Following placement, the system 700, 720 may also include performing or assisting with closure of the surgical site, such as suturing or staple placement. For example, the end effector may include mechanisms for advancing suture thread, forming knots, cutting suture strands, or deploying pre-tied knots or clips, thereby enabling automated or semi-automated tissue closure. This approach may allow precise and accurate positioning of the regenerative sleeve, while minimizing injury to surrounding tissues and risk to the patient.

Although several embodiments of the invention have been disclosed in the foregoing specification, it is understood that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. It is further envisioned that features from one embodiment may be combined or used with the features from a different embodiment described herein. Moreover, although specific terms are employed herein, as well as in the claims which follow, they are used only in a generic and descriptive sense, and not for the purposes of limiting the described invention, nor the claims which follow. The entire disclosure of each patent and publication cited herein is incorporated by reference in its entirety, as if each such patent or publication were individually incorporated by reference herein. Various features and advantages of the invention are set forth in the following claims.

What is claimed is:

1. A system for treating a spinal cord injury of a patient comprising:
a regenerative device configured to be implanted adjacent to an injured segment of a spinal cord of a patient, the regenerative device including a body configured to at least partially encircle the injured segment of the spinal cord, and the regenerative device comprises a porous structure configured to stabilize a local microenvironment surrounding the injured segment of the spinal cord,
wherein the regenerative device is coupled to an electrical stimulation device, for stimulating growth in nerve tissue and reducing scar tissue formation.

2. The system of claim 1, wherein the regenerative device is a sleeve, collar, cuff, wrap, or scaffold configured to circumferentially surround the spinal cord.

3. The system of claim 1, wherein the porous structure comprises a mesh, lattice, scaffold, fabric, or bioengineered material.

4. The system of claim 1, wherein the regenerative device includes longitudinal microchannels oriented along a longitudinal axis of the spinal cord.

5. The system of claim 1, wherein the regenerative device is configured to be positioned external to a dura mater surrounding the spinal cord.

6. The system of claim 1, wherein the regenerative device is configured to be positioned intradurally adjacent to the spinal cord.

7. The system of claim 1, wherein the regenerative device includes one or more biologic agents configured to promote neural regeneration.

8. The system of claim 7, wherein the biologic agents include growth factors, stem cells, blood, or therapeutic drugs.

9. The system of claim 1, wherein the regenerative device includes one or more suture tabs having suture holes configured to receive sutures to secure the device to local anatomy.

10. A system for treating a spinal cord injury of a patient comprising:
a regenerative device configured to be positioned adjacent to an injured segment of a spinal cord of a patient, the regenerative device including one or more segments configured to at least partially encircle the spinal cord; and
a securing member configured to connect ends of the one or more segments to maintain the regenerative device around the spinal cord,
wherein the regenerative device is coupled to an electrical stimulation device, for stimulating growth a nerve tissue and reducing scar tissue formation.

11. The system of claim 10, wherein the regenerative device includes a single segment and the ends of the segment are joined together by the securing member.

12. The system of claim 10, wherein the regenerative device includes a first segment and a second segment configured to form a circumferential sleeve when connected.

13. The system of claim 10, wherein the securing member includes a clip configured to couple the ends of the one or more segments.

14. The system of claim 10, wherein the securing member is configured to permit adjustment of a diameter of the regenerative device when positioned around the spinal cord.

15. The system of claim 10, wherein the one or more segments include reinforced edges to support connection by the securing member.

16. A method of treating a spinal cord injury comprising:
accessing an area near a spinal cord of a patient; and
implanting a regenerative device adjacent to an injured segment of the spinal cord such that the regenerative device at least partially encircles the spinal cord, the regenerative device including a porous structure configured to stabilize a local microenvironment surrounding the injured segment and/or promote axonal regrowth across the injury site,
wherein the regenerative device is coupled to an electrical stimulation device, for stimulating growth a nerve tissue and reducing scar tissue formation.

17. The method of claim 16 further comprising positioning the regenerative device intradurally adjacent to the spinal cord.

18. The method of claim 16 further comprising positioning the regenerative device external to dura mater surrounding the spinal cord.

19. The method of claim 16 further comprising promoting axonal growth along microchannels in the regenerative device oriented along a longitudinal axis of the spinal cord.

20. The method of claim 16 further comprising adding one or more biologic agents to the regenerative device to promote neural regeneration or support neural repair.
